(19) United States
(12) Patent Application Publication
(10) Pub. No.: US 2006/0110425 A1
(43) Pub. Date: May 25, 2006

(54) Leech extracts for stents

(75) Inventors: Jacques Latrille, Eysines (FR); Quennady I Nikonov, Moscow (RU)

Correspondence Address: FOLEY AND LARDNER LLP;SUITE 500, 3000 K STREET NW, WASHINGTON, DC, 20007, US

(73) Assignee: RICARIMPEX

(21) Appl. No.: 10/523,937

(22) PCT Filed: Aug. 6, 2003
(86) PCT No.: PCT/FR03/02473

§ 371 Date: Nov. 16, 2005

(30) Foreign Application Priority Data
Aug. 7, 2002 (FR) 02/10042

(51) Int. Cl. A61K 38/46; A61F 2/00; A61K 9/127

(52) U.S. Cl. 424/423; 424/450; 424/94.6

(57) ABSTRACT

The invention relates to a stable destabilase complex in the form of a monomer, which is capable of aggregating into a polymer destabilase complex forming a liposome and which can be obtained from medicinal leeches using the following method comprising: affinity chromatography with 6-keto-prostaglandin antibodies which are immobilised on a suitable column; and high ionic strength elution of the destabilase complex, said complex having an antithrombin activity of at least 700 ATU/mg, a plasma recalcification time of at least 800 APC/mg, a fibrinolytic activity of at least 40 mm²/mg and an immunomodulating activity.

The invention relates to the production of biologically active substances with cosmetic or pharmaceutical properties in the form of liposomes. More precisely, these liposomes are natural liposomes extracted from medicinal leeches. These natural liposomes have both anticoagulating and immunomodulating properties.

Compounds with anticoagulating properties such as heparin, and compounds with immunomodulating properties are already known. These types of compounds are used for therapeutic purposes, and are for example administered through physically acceptable supports called “stents”.

These stents are endoprostheses frequently used in cardiovascular surgery to be implanted into a blood vessel, and particularly a coronary artery or a peripheral artery. It is known that atheromatous vascular diseases corresponding to narrowing of the arteries can be treated by expansion cuff techniques, techniques named angioplasties. The cuff is inserted into the artery and inflated to a pressure at which it crushes the deposit of atheromas. However, these techniques are not always sufficient, and consequently it is known to implant in the blood vessel in the area treated by angioplasty, an endoprosthesis, more frequently called a stent, is implanted typically consisting of a metallic support acting as tutor once it has been inserted inside the artery at the treated zone. For example, such stents are clamped on the cuff, in a so-called rest position, before the cuff is inserted into the blood vessel. Once the stent has been transferred to the implantation area inside the blood vessel, it is deployed by expanding the cuff so as to come into contact with the wall of the blood vessel to be widened.

It is known that stents can be covered with therapeutically active substances, particularly intended to act in the implantation area.

Very many stent devices are known in prior art. For example, methods and equipments for releasing active substances from stent type devices are described in U.S. Pat. Nos. 6,096,070; 5,824,049; 5,624,411; 5,609,629; 5,569,463; 5,447,724; and 5,464,650. The use of stents to release medicine is described for example in documents WO 01/01957, U.S. Pat. Nos. 6,099,561; 6,071,305; 6,063,101; 5,997,468; 5,980,551; 5,980,566; 5,972,027; 5,968,092; 5,951,586; 5,893,840; 5,891,108; 5,851,231; 5,843,172; 5,837,008; 5,769,883; 5,735,811; 5,700,286; 5,679,400; 5,649,977; 5,637,113; 5,591,227; 5,551,954; 5,545,208; 5,500,013; 5,464,450; 5,419,760; 5,411,550; 5,342,348; 5,286,254; and 5,163,952. Stent cladding methods are described particularly in U.S. documents Pat. Nos. 6,409,716; 6,464,893 and 5,356,433.

However, prior art does not describe any substances that are capable of forming a cladding for stents in the form of liposome, and with anticoagulating and immunomodulating properties. Two separate medicines have to be administered to the patient according to prior art, namely an anticoagulating substance and an immunomodulating substance.

Remember that liposomes are advantageously very useful as vectors both for apolar and polar pharmaceutically active compounds. This form of liposome can thus be used to administer these two types of compounds on the same site.

The invention is intended to overcome the disadvantages of prior art, and particularly to obtain a composition with both anticoagulating and immunomodulating properties, capable of being associated with equipment such as stents, and in the form of liposomes so as to obtain an appropriate release of the substance.

The inventors have successfully obtained such a substance from medicinal leeches.

A method of obtaining a destabilase complex called a prototype is known in prior art, making use of an affinity chromatography step on an insoluble support in water (for example CNBr-activated agarose) carrying immobilised lysine (REF). The extract is transported by a buffer with pH lower than neutral pH, the pH is neutralised, and dialysis and freeze-drying steps are then carried out. However, elution of the final product from the immobilised lysine using a buffer with a low pH less than 9 cannot produce a destabilase complex suitable for the required storage and the sufficient enzymatic activity that is required. The destabilase molecule extracted by this process is in the form of an unstable monomeric enzymatic complex which is subject to a conformation change so that it loses its capacity to form an enzymatic complex in polymeric form. The enzymatic complex in monomer form no longer has the capacity to structure itself into a polymer that will organise itself into a liposome, as required. Known complexes (prototypes) produced by medicinal leeches form a complex of hirudin, prostaglandin, destabilase and blood plasma kallikrein inhibitors in the ratio 1:1:1:1.

Abstract XP002237190 of patent application RU 2 129 429 C describes a process for isolating prostaglandin complexes originating from medicinal leeches. The process requires purification of the mix by affinity chromatography with anti 6-cetoprostaglandin-F1 antibodies immobilised on a support insoluble in water.

The document by Nikonov Gl et al (Destabilase complexes—natural liposome produced by medicinal leeches hirudo medicinalis” Fundamental & Clinical Pharmacology, Elsevier, Paris, F R, vol. 13, No. 1, 1999, pages 102-106) describes a process for producing natural liposomes destabilase complexe-based obtained from medicinal leeches. The following procedure is carried out to obtain destabilase complexes with micella type properties:

extraction from leech heads using organic solvents,

affinity chromatography comprising anti-ceto-PFG antibodies immobilised on a CNBr-activated sepharose 4B substrate.

The eluate is analysed by electrophoresis. The fraction obtained is composed of a destabilase monomer aggregate and has micellar protein properties and is represented by a stable lipid-protein complex.

Destabilase monomers may take the form of a destabilase complex polymer forming a liposome. The said liposomes have the capacity to quickly penetrate cellular membranes and have an antithrombotic activity.

Patent application WO 92 02206 describes freeze dried native collagen sheets including cosmetic formularies for the treatment of rosacea. Active ingredients of the cosmetic formulation may include hirudin, an anticoagulating protein originating from leeches.

Patent application WO 98 16604 describes washing compositions, preferably detergent compositions for washing products, comprising an iso-peptidase type enzyme with the capacity of catalytically breaking bonds between glutamine and lysine.

Patent application WO 01 47572 describes a method of inhibiting restenosis of a blood vessel comprising:

supply of a device comprising an active compound with an antithrombotic and anti-inflammatory activity, and

implantation of the device in the blood vessel to inhibit restenosis of the said blood vessel.

The inventors set themselves the objective of obtaining a complex of stable destabilase with better biological activities than compositions according to prior art.

The inventors successively obtained a monomeric destabilase complex that is stable and capable of aggregating into polymers organising themselves into liposome.

Thus, the purpose of a first aspect of the invention is a stable destabilase complex in monomer form, capable of aggregating into a polymer destabilase complex forming a liposome, and that can be obtained from medicinal leeches by a process comprising:

an affinity chromatography with the said antibodies of 6-keto-prostaglandin immobilised on a suitable column;

elution with a high ionic strength of the destabilase complex,

the said complex having an antithrombin activity of at least 700 ATU/mg, a plasma recalcification time of at least 800 APC/mg, a fibrinolytic activity of at least 40 mm²/mg, and an immunomodulating activity.

The purification process is made by chromatography, preferably affinity chromatography. This process comprises:

an affinity chromatography with the said antibodies of 6-keto-prostaglandin immobilised on a suitable column, preferably made of 6-keto-PGF1_{α};

elution with a high ionic strength of the destabilase complex.

The said destabilase complex obtained by this process is in monomer form and includes destabilase and at least one compound in the hirudin, prostaglandin, kallikrein inhibitor group.

The action of the anti 6-keto-PGF1_{α} antibody and Sepharose lysine can also be combined.

The liposome destabilase complex has very useful pharmaceutical properties, like the monomeric destabilase complex, namely typically an antithrombin activity of at least 500, preferably at least 600 or 700 ATU/mg, a plasma recalcification time of at least 800 APC/mg, and a fibrinolytic activity of at least 40 mm²/mg. The antithrombic and thrombolytic action is significantly better than with the control and the prototype.

The invention also relates to a pharmaceutical composition comprising a liposome destabilase complex according to the invention and a pharmaceutically acceptable vehicle.

The term pharmaceutically acceptable vehicle is used to denote a non-toxic, solid or liquid diluting or encapsulating material that does not react on the active compound so as to reduce its efficiency.

The invention also relates to a cosmetic composition comprising the liposome destabilase complex according to the invention.

The invention also relates to the liposome destabilase complex as a medicine, and use of a monomer or polymer liposome distabilase complex for preparation of a medicine with an anticoagulating and immunomodulating activity.

The invention also relates to a device for purification of a destabilase complex from medicinal leeches, comprising an affinity column charged with anti 6-keto-prostaglandin antibodies.

The invention also relates to an implantable medical prosthesis in which at least part of the prosthesis is covered with a cladding, the said cladding including liposome distabilase complex according to the invention.

In one preferred embodiment, the implantable prosthesis is a stent support.

Other purposes and advantages of the invention will become clear in the following detailed description.

The purification process for the stable destabilase complex according to the invention comprises an affinity chromatography step using the 6-keto-PGF1_{α} (6-keto-prostaglandin F_{1α}) antibodies immobilised on a support insoluble in water. The principle of immuno-affinity chromatography is known to those skilled in the art and is based on the specificity of mono or polyclonal antibodies to capture specific proteic antigens from complex natural extracts. However, the inventors have successfully and very surprisingly obtained a destabilase complex with conserved anticoagulating and immunomodulating biological activities capable of organising themselves into liposomes.

The antibodies are coupled to a solid chromatographic phase, typically agarose, through covalent bonds (for example cyanogen bromide CNBR) or other chemical couplings targeting the amino, hydroxyl, carboxyl or sulphydryl groups of immunoglobulins so as to form a solid matrix. The solid phase coupled to antibodies is then placed in an affinity chromatography column. The mix of the target antigen and contaminants is diluted in a bond buffer and is then applied to the column. Non-adsorbed contaminants are eliminated by washing with different buffers. Elution of the target proteic antigen is then obtained for example by using extreme pH conditions, changes of ionic strengths.

Hirudin and kallikrein inhibitors have an activity only in the aqueous phase. Destabilase has an activity only in the non-aqueous phase.

The stable destabilase complex obtained by the inventors has hydrophobic properties dependent on the prostaglandin component, and hydrophilic properties dependent on the liposome polypeptides.

The monomer form of the destabilase complex has a molecular weight of 25 kDa, is stable, and has an aggregation capacity that leads to the formation of liposomes. It is obtained from total leech extracts, or particularly a fraction of secretions from salivary glands or blood of the digestive tube of the leech. After purification by affinity chromatography, the destabilase complex obtained includes the following compounds: hirudin, prostaglandin (substance similar to a prostacyclyn), kallikrein inhibitor, destabilase. The destabilase complex has the following properties: antithrombotic activity by hirudin, increase in the blood plasma recalcification time by kallikrein inhibitors, blockage of adhesion and platelet aggregation by substances similar to prostacyclins, dissolution of fibrins stabilised by destabilase.

These properties of the destabilase complex determine its antithrombotic, thrombolytic efficiency (2.5 times greater than the prototype), and its immunomodulating activity, and its hypotensive activity that is completely lacking in the prototype.

We will now present methods of measuring the biological activity used for the purified leech extract and in liposome form, followed by four example embodiments demonstrating the anticoagulating and immunomodulating activity of the destabilase complex according to the invention.

1/ The antithrombin activity is determined by an extension in the time of precipitation of fibrinogen by thrombin. The time for formation of a precipitate in a system including 0.2 ml of a solution of 0.3% fibrinogen and 0.1 ml of the destabilase complex after fixation of 0.1 ml of a solution of thrombin containing one unit of thrombin activity, is determined. The hirudin activity is expressed in international antithrombin units (ATU NIH).

2/ The blood plasma recalcification time is determined in a system comprising 0.1 ml of citric blood plasma and 0.1 ml of destabilase complex after fixation of 0.1 ml of a 0.025 M solution of CaCl₂. Doubling this parameter corresponds to one APC unit.

3/ The content in prostaglandins is determined using a radio immuno-test X for 6-keto-PGF1_{α} obtained from the “Amersham” Company.

4/ The antithrombotic action of the destabilase complex is determined on rats using Wessler's thromboformation method (5). The thromboformation blocking level is evaluated with respect to the control. This is done by studying the results following 4 hours separation between injection of the destabilase complex and injection of human blood serum activated by cold. This degree of blocking is expressed as a percent of the control (equal volume of a normal saline solution).

5/ The hypotensive action is determined following oral administration of 0.2 ml of a destabilase complex in an SHR line of rats (spontaneously hypertensive). The initial pressure was 165±5 mm. After 10 hours of analysis, the blood pressure in the rat was measured in the tail vein. The hypotensive efficiency level is expressed as a percent of the control (equal administered volume of a normal saline solution).

6/ The cytophage activity of neutrophiles is determined by the known Chernushenko method (6). Research was done on twenty pubescent rats. An aqueous solution of destabilase complex was injected daily intra-veinously for ten days at a dose of 0.5 ml (n=10). An equal volume of a 0.85% NaCl solution was injected in control animals (n=10). After the appropriate time, blood was collected in the animals and the cytophage activity of neutrophiles was studied. A phagocytosis index was defined; a cytophage index (CI) and a phagocytosis percent (Ph).

7/ The influence on cellular activity was studied by measuring the inhibition of the activity of constituents of the complement system. A method of defining the haemolytic activity of dilute human serum (7) was also used.

The results were as follows:

### EXAMPLE 1

The protocol is as follows:

take ten medicinal leeches (total 12 g), homogenise them, and mix with distilled water until the total homogenised volume is 24 ml (volume ratio 1:1), then grind and collect the supernatant.

Put the supernatant of the homogenate on an agarose column comprising anti 6-keto PGF1. immobilised antibodies.

The elution is obtained by a buffer comprising 0.2 M of glycine with 0.15 M HCl and 0.5 M NaCl. The eluate volume is 35 ml.

As can be seen in table 1, the final product has a fibrinolytic, antithrombin activity, increases the recalcification time, contains prostaglandin and in addition to the strong antithrombolytic potential, has a hypotensive action reducing the blood pressure by 25% (bringing it almost back to normal).

### EXAMPLE 2

5 ml of a secretion of leech salivary glands was added into an agarose column with anti 6-keto-PGF1_{α} immobilised antibodies. The elution is obtained using a 0.5 M phosphate buffer with pH 6.4. The eluate volume is 10 ml.

As shown in table 1, the final product has an antithrombin fibrinolytic activity, increases the recalcification time, contains prostaglandin and in addition to the strong antithrombic and thrombolytic potential, has a hypotensive action reducing the blood pressure by 25% (bringing it almost back to normal). The destabilase complex increases the cytophage index and the phagocytosis percentage (table 1), demonstrating the immunostimulative action.

### EXAMPLE 3

10 ml of blood from the digestive tube of medicinal leeches (three months after their last meal) is placed in a column of L-Glutamine-Sepharose. The elution is obtained with a 1 M solution of KCl. The eluate volume is 20 ml. As shown in table 1, the final product has an antithrombin fibrinolytic activity, increases the recalcification time, contains prostaglandin and in addition to the strong antithrombic and thrombolytic potential, has a hypotensive action reducing the blood pressure by 25% (bringing it almost back to normal). The destabilase complex increases the cytophage index and the phagocytosis percentage (table 1), demonstrating the immunostimulative action.

### EXAMPLE 4

The protocol is as follows: take twenty medicinal leeches (total 21 g), extracting the front part of the animal, homogenising it and collecting the aqueous extract. 10 ml of the extract are placed in a L-Lysine-Sepharose column. The elution is obtained with a 1 M solution of KCl. The eluate volume is 20 ml.

As shown in table 1, the final product has an antithrombin fibrinolytic activity, increases the recalcification time, contains prostaglandin and in addition to the strong antithrombic and thrombolytic potential, has a hypotensive action reducing the blood pressure by 25% (bringing it almost back to normal). The destabilase complex increases the cytophage index and the phagocytosis percentage (table 1), demonstrating the immunostimulative action.

TABLE 1
definition of leech extract activities

| | | Examples | | | | |
|---|---|---|---|---|---|---|
| Index | Control | No. 1 | No. 2 | No. 3 | No. 4 | Prototype |
| Antithrombin activity ATU/mg | 74 ± 6 | 835 ± 45 | 875 ± 68 | 775 ± 44 | 865 ± 34 | 250 ± 20 |
| Blood plasma recalcification time APC/mg | 115 ± 14 | 1025 ± 86 | 930 ± 52 | 950 ± 50 | 1030 ± 72 | 438 ± 35 |
| Fibrinolytic activity Mm²/mg | 9 ± 4 | 58 ± 5 | 49 ± 5 | 52 ± 7 | 50 ± 6 | 26 ± 5 |
| Prostaglandins ηg/mg | 729 ± 35 | 955 ± 20 | 800 ± 34 | 870 ± 54 | 900 ± 44 | 85 ± 13 |
| % antithrombotic action | 60 ± 4 | 100 | 100 | 100 | 100 | 65 ± 5 |
| % thrombolytic action | 10 ± 5 | 80 ± 5 | 75 ± 5 | 70 ± 5 | 75 ± 5 | 10 ± 5 |
| % hypotensive action | 8 ± 3 | 25 ± 5 | 25 ± 5 | 20 ± 5 | 25 ± 5 | 0 |
| Cytophage index | 1.8 ± 0.7 | 6.4 ± 1.1 | 5.9 ± 0.8 | 6.3 ± 1.0 | 6.0 ± 0.7 | 0.8 ± 0.3 |
| % phagocytosis | 45.4 ± 6.3 | 75.4 ± 6.0 | 71.4 ± 7.3 | 68.5 ± 8.0 | 69.7 ± 6.6 | 35.7 ± 6.7 |
| Immunomodulating activity | − | + | + | + | + | − |

Those skilled in the art can use a large number of different methods for application of the purified extract on the stents. Thus, the inventors have obtained very many different types of destabilase complex support stents. For example, a stent will have an external polymeric surface on which a gelatinous matrix will be placed, the matrix including the destabilase complex in the form of liposomes. According to one embodiment, the polymeric surface will be bonded by covalent bonds with the gelatinous matrix. For example, the polymeric gel may be between 10 to 50 μm thick in the non-compressed state. For example, this gel may be chosen from the group composed of polycarboxylic acids, cellulose polymers, gelatine, polyvinylpyrrolidone, maleic anhydride polymers, polyamides, polyvinylic alcohols, polyethylene oxides, and polyacrylic acid.

1. Stable destabilase complex in monomer form, capable of aggregating into a polymer destabilase complex forming a liposome, that can be obtained from medicinal leeches by a process comprising:
an affinity chromatography with the said 6-keto-prostaglandin antibodies immobilised on a suitable column;
elution with high ionic strength of the destabilase complex,
characterised in that it has an antithrombin activity of at least 700 ATU/mg, a plasma recalcification time of at least 800 APC/mg, a fibrinolytic activity of at least 40 mm²/mg, and an immunomodulating activity.

2. Pharmaceutical composition comprising a destabilase complex according to claim 1 and a pharmaceutically acceptable vehicle.

3. Cosmetic composition comprising a destabilase complex according to claim 1.

4. Destabilase complex according to claim 1 as a medicine.

5. Use of a destabilase complex according to claim 1 for preparation of a medicine with an anticoagulating and immunomodulating activity.

6. Implantable medical prosthesis, in which at least part of the prosthesis is covered with a cladding, the said cladding comprising a destabilase complex according to claim 1.

7. Implantable prosthesis according to claim 6, characterised in that it is a stent support.
